(19) United States
(12) Patent Application Publication
(10) Pub. No.: US 2021/0069566 A1
(43) Pub. Date: Mar. 11, 2021

(54) VISUAL RHYTHMIC GUIDE DEVICE FOR SWIMMING TRAINING

(71) Applicants: Wen-Shen KO, Taipei City (TW); Chih-Han KO, Taipei City (TW); Wang-Hsiang KO, Taipei City (TW)

(72) Inventors: Wen-Shen KO, Taipei City (TW); Chih-Han KO, Taipei City (TW); Wang-Hsiang KO, Taipei City (TW)

(21) Appl. No.: 16/885,583

(22) Filed: May 28, 2020

(30) Foreign Application Priority Data
Sep. 6, 2019 (TW) 108211874

(51) Int. Cl. A63B 69/12

(52) U.S. Cl. CPC A63B 69/12; A61B 5/1124

(57) ABSTRACT

The objective of the invention is to provide a visual rhythmic guide device for swimming training capable of emitting rhythmical flashlights with a given frequency to lead the tempo of movements such as arm stroking and leg kicking for a swimmer. To achieve the objective, the device comprises a waterproof body to house components including a control circuit board, a timer, a gravity sensor (G-Sensor), a LED light source, a screen, a battery, a connection port and a set of input keys. When the device is given a frequency by a swimmer through the input keys, the LED light source will be driven to emit rhythmical flashlights in accordance with the given frequency. The swimmer is able to wear the device on anywhere of the body as long as the rhythmical flashlights can be sensed to adjust and practice the tempo of swimming movements.

### BACKGROUND OF THE INVENTION

### (a) Field of the Invention

The present invention relates to a visual rhythmic guide device for swimming training and, more particularly, to a device able to send out flashlights with a given frequency for a period of time to lead the tempo of movements such as arm stroking and leg kicking for a swimmer in a training practice.

### (b) Description of the Prior Art

Swimming is an important category of international sport competition. In addition, taking a swimming exercise is helpful for people to increase body strength, to improve physique and to keep healthy. As a result, swimming is a very popular sport all over the world, and many people are eager for useful tools and continuous training to improve their swimming skills.

An audio-instrument for swimming practice is currently available in the market, which generates tick sounds with a particular rate like a metronome for music. A user can place the audio instrument inside a swimming cap, hang it on the head strap or the nose strap of a goggle or wear it on ears. The user is able to adjust his movement of limbs to match the tick sounds during a swimming practice.

However, sound performs very differently in the medium of water than in the medium of air. The tick sounds generated by the audio instrument for swimming practice can be easily interfered with by the environmental noises in a swimming pool. In addition, the noises made by the swimmer himself when splashing water always block the tick sounds of the audio instrument, which may disturb the swimmer's tempos during a training practice. In order to hear the tick sounds clearly, it is necessary to place the audio instrument as near to the ears as possible, and wearing the audio-instrument on ears often causes pain and discomfort to the ears.

Therefore, the audio-instrument at present is not well designed enough, and further improvements of the disadvantages are required.

### SUMMARY OF THE INVENTION

Based on years of experience in designing, developing and manufacturing relevant products, the inventor of the present application has accomplished the improvements of the described disadvantages after strenuous experiments and careful evaluations. The objective of the invention is to provide a visual rhythmic guide device for swimming training which is able to emit flashlights with a preset frequency for a given period of time to lead the tempo of movements such as arm stroking and leg kicking for a swimmer during a training practice.

To achieve the described objective, the visual rhythmic guide device for swimming training of the invention mainly comprises a waterproof body, a control circuit board, a timer, a gravity sensor (G-Sensor), a LED (Light Emitting Diode) light source, a screen and a set of input keys. The control circuit board is enclosed inside the waterproof body to drive and integrate every functions and operations of the device. The timer inside the waterproof body is embedded in the control circuit board to provide all time parameters required to operate the control circuit board. The gravity sensor (G-Sensor) is electrically connected to the control circuit board inside the waterproof body to detect the turning movement of a swimmer so as to count the number of laps. The LED light source is located at the lower portion of the waterproof body, which is electrically connected to the control circuit board to emit rhythmical flashlights according to a group of preset frequency and time duration. The screen is provided on the upper portion of the waterproof body, which is electrically connected to the control circuit board to display the operational information being processed. The input keys are located at the external portion of the waterproof body, which are electrically connected to the control circuit board to input all parameters required by the device, such as the frequency and the time duration of the rhythmical flashlights.

When the visual rhythmic guide device for swimming training is used in a training practice, the device can be placed on anywhere of the user's body as long as the rhythmical flashlights emitted from the LED light source of the device can be sensed by the user. Therefore, the user is able to adjust and practice his movements of limbs by following the tempo of the rhythmical flashlights generated by the device according to a preset frequency during a given period of time to perform a rhythmic swimming training.

To enable a further understanding of the objective and the technological methods of the invention herein, the brief description of the drawings below is followed by the detailed description of the preferred embodiments.

### BRIEF DESCRIPTION OF THE DRAWINGS

FIG. 1 is an external view showing the appearance of the visual rhythmic guide device for swimming training of the invention.

FIG. 2 is a block diagram to explain the arrangement of all components of the visual rhythmic guide device for swimming training of the invention.

### DETAILED DESCRIPTION OF THE PREFERRED EMBODIMENTS

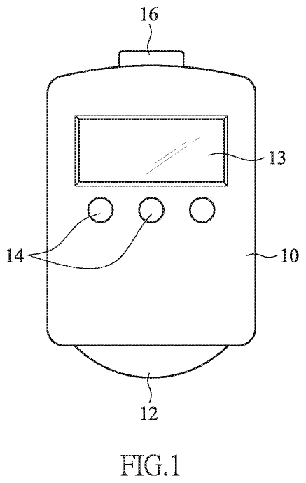

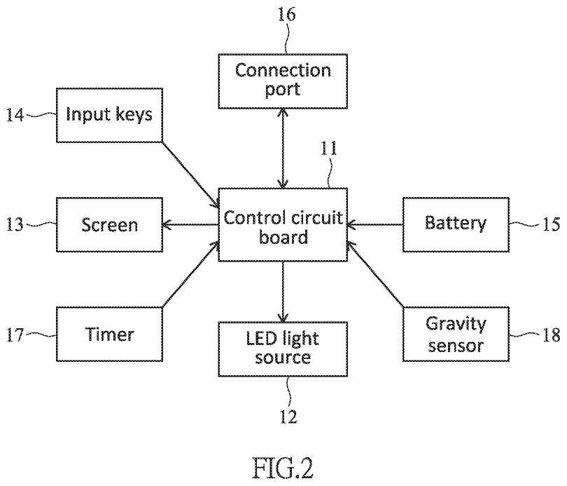

The present invention provides a visual rhythmic guide device for swimming training. Referring to FIG. 1 and FIG. 2, the visual rhythmic guide device for swimming training comprises a waterproof body 10, a control circuit board 11, a timer 17, a gravity sensor (G-Sensor) 18, a LED light source 12, a screen 13, a set of input keys 14, a battery 15 and a connection port 16.

The waterproof body 10 is hollow with a water proof supporting case as the outer enclosure of the device, capable of housing all components of the device in sealed in the water proof supporting case.

The control circuit board 11 is enclosed inside the waterproof body 10 for driving and integrating all functions and operations of the device.

The timer 17, provided inside the waterproof body 10, is embedded in the control circuit board 11 to provide all time parameters required to operate the control circuit board 11.

The gravity sensor 18, provided inside the waterproof body 10, is electrically connected to the control circuit board 11 to detect the turning movements of a swimmer and to calculate the numbers of laps accordingly.

The LED light source 12, located at the lower portion of the waterproof body 10, is electrically connected to the control circuit board 11 to emit rhythmical flashlights according to a group of frequency and time duration given by the user.

The screen 13, located at the upper portion of the waterproof body 10, is electrically connected to the control circuit board 11 to display the operational information being processed for the user. A LCD panel (liquid crystal screen) is applied to the device of the invention.

The input keys 14, located at the external portion of the waterproof body 10, are electrically connected to the control circuit board 11 enabling the user to set the frequency and the time duration of the rhythmical flashlights of the LED light source 12 by pressing the input keys 14.

The battery 15, provided inside the waterproof body 10, is electrically connected to the control circuit board 11 to supply electricity to every components of the device through the control circuit board 11. In addition, the battery 15 is chargeable.

The connection port 16 such as an USB (Universal Serial Bus) connector applied to the invention, located at the upper portion of the waterproof body 10, is electrically connected to the control circuit board 11. The battery 15 is able to be charged by wiring the connection port 16 with an external power supply (not shown in the drawings).

When the visual rhythmic guide device for swimming training is used in a training practice, the device can be put on many places of the user's body such as the nose bridge between two lenses of a goggle, the forehead, the chest or the abdomen as long as the rhythmical flashlights emitted from the LED light source 12 can be sensed or glimpsed. The user is able to practice the swimming tempo of arm stroking and leg kicking by following the lead of the rhythmical flashlights accordingly.

On comparison with the prior art, the visual rhythmic guide device for swimming training has the following advantages:

1. Visible rhythms are used to guide the tempo of swimming movements such as arm stroking and leg kicking.

2. Rhythmical flashlights are not easily interfered with by the environmental noises in a swimming pool and the noises of splashing water made by the swimmer himself.

3. Wearing the device on a suitable place of the swimmer's body causes less pain or discomfort.

In summary, the visual rhythmic guide device for swimming training of the invention involves a novel structure which is not disclosed in any publication. The same product commercially available is not found in the market. In addition, the invention provides unique characteristics and functions which cannot be found in the prior art. Therefore, the patent application is submitted accordingly.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A visual rhythmic guide device for swimming training comprising:
a waterproof body, being hollow with a water proof supporting case as the outer enclosure of the device to house all components in sealed;
a control circuit board, enclosed inside the waterproof body to drive and integrate all functions and operations of the device;
a timer, enclosed inside the waterproof body and embedded in the control circuit board to provide all time parameters required to operate the control circuit board;
a gravity sensor, provided inside the waterproof body and electrically connected to the control circuit board to detect the turning movements of a swimmer and to calculate the numbers of laps accordingly;
a LED (Light Emitting Diode) light source, located at the lower portion of the waterproof body and electrically connected to the control circuit board to emit rhythmical flashlights according to a group of frequency and time duration given by the user;
a screen, located on the upper portion of the waterproof body and electrically connected to the control circuit board to display the operational information being processed; and
a set of input keys, located at the external portion of the waterproof body and electrically connected to the control circuit board enabling the user to input the frequency and the time duration of the rhythmical flashlights by pressing the input keys.

2. The visual rhythmic guide device for swimming training of claim 1, wherein the waterproof body is provided with a battery electrically connected to the control circuit board to supply electricity to every components of the device through the control circuit board.

3. The visual rhythmic guide device for swimming training of claim 2, wherein the battery is chargeable.

4. The visual rhythmic guide device for swimming training of claim 1, wherein the water proof body is provided with a connection port electrically connected to the control circuit board to charge the battery by wiring the connection port to an external power supply.

5. The visual rhythmic guide device for swimming training of claim 4, wherein the connection port is an USB (Universal Serial Bus) connector.

6. The visual rhythmic guide device for swimming training of claim 1, wherein the screen is a LCD (liquid crystal display) panel.

7. The visual rhythmic guide device for swimming training of claim 1, wherein the device is able to produce rhythmical flashlights with a given frequency for a user to practice the tempo of swimming movements by following the lead of the rhythmical flashlights.
